SEQUENCE LISTING (4 sequences)

SEQ ID NO: 1 - protein, 562 aa, synthetic construct
MSSPGPSQPPAEDPPWPARLLRAPLGLLRLDPSGGALLLCGLVALLGWSWLRRRRARGIPPGPTPWPLVGNFGHVLLPPFLRRRSWLSSRTRAAGIDPSVIGPQVLLAHLARVYGSIFSFFIGHYLVVVLSDFHSVREALVQQAEVFSDRPRVPLISIVTKEKGEREVVGCGYADAADESPGVVFAHYGPVWRQQRKFSHSTLRHFGLGKLSLEPKIIEEFKYVKAEMQKHGEDPFCPFSIISNAVSNIICSLCFGQRFDYTNSEFKKMLGFMSRGLEICLNSQVLLVNICPWLYYLPFGPFKELRQIEKDITSFLKKIIKDHQESLDRENPQDFIDMYLLHMEEERKNNSNSSFDEEYLFYIIGDLFIAGTDTTTNSLLWCLLYMSLNPDVQEKVHEEIERVIGANRAPSLTDKAQMPYTEATIMEVQRLTVVVPLAIPHMTSENTVLQGYTIPKGTLILPNLWSVHRDPAIWEKPEDFYPNRFLDDQGQLIKKETFIPFGIGKRVCMGEQLAKMELFLMFVSLMQSFAFALPEDSKKPLLTGRFGLTLAPHPFNITISRR

SEQ ID NO: 2 - DNA, 3939 bp, synthetic construct
  source 1..3939: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggtgagcgggaggtcgtgggctgtgggtacgcggatgccgcggatgagtctccaggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 3 - DNA, 3712 bp, synthetic construct
  source 1..3712: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctgccaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggtgagcgggaggtcgtgggctgtgggtacgcggatgccgcggatgagtctccaggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctggcttttaaagacctatgacaacatgaaagtggtgttcagcctggaatgctttgtcagagatgggtgtggatttaggttatactgggggagaacttttctcagcacagattctatgccagcttctttgggcttgttctgtcactatctttttgtttatgattttagtttttactttttgtagatgtgggatgaagtggactctgtcgtgtatattgaggaaaaaagaaattataattttaaaaaatcccttgtaggattattatctaaatttatatgtctaacttctactacaactacaggaacagtgagccttgctacttctttagtagcttcttggcagaattcctttctactgagttatttgcaaagatgcagctctacctttttacttaaggcctgaatggtgagcatggggattttgatactgggactcatcaggaaaggattctgctttcaaactatactgaacattcctgtcctagacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 3840 bp, synthetic construct
  source 1..3840: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcaagactgaaagggaagcccttatcaggaaagccattatctgagaagataaaataaagtaatactttgtttttaaagtattatacaattcatatctaatatttcaacctcagcctttcaagtgttgtctaacaccaataataatattgccacccctccctcaaccggttatcttcttttcatatttctagctacttaaatccaccaagcaaattctggtttcgctatttctggatttaagtacattttctaatgcacccaggccacacgcaggaatggacacagctttcgctctccgcataagttttctagcccagggcaagctctcagaacgtgactcctcgtgcctcgctgcccgcccctcgctccccccactcccgccatcatcccttccgcagagttggctgccaagccgacacccacagattctgcattccacggcttcccagcatcctgctgtctctgcgtggaggaaagggtgctgcgaatcccagtttagataaggaaccccgtcttttcactactacaccgaagcatgcaggaatagggcgttgaccccattctcccagctcagggcacaggtgctaaggccgacccatctcacgggcctgttcaccagtttcccacactcagtacagatcctaacacggtttatgcacttcataaccatgcgtttgtgtttgtcaactgacaaacgagtgataatgacaaacgaatgacacaagggaaaccaacagccaatcagagcgcaaggttgtgttgccagcatcccagtttcagggacagtcaccgccaagcccgaccctgcaaagggtcgtaaccggacttcggggcaaacttcaggtcccccgcctgctagcccgccaaccccctccgccaggcagccggcgcgcctccaggttccgcccaggcgtcgctgagtggcggcggcggggagaatgcgcgcggcgcagccaatccgggggcgttcctacaccccctgcccgcccccgaccttccagagcagagcaggacactggcgccgcgggtcaggcagctgcgtgcgcgtctcctccaggcagcaaggggaacccgaggccgccggcgcccaagtttgtacaaaaaagcaggctgccaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggtgagcgggaggtcgtgggctgtgggtacgcggatgccgcggatgagtctccaggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgc

(19) United States
(12) Patent Application Publication
(10) Pub. No.: US 2023/0211014 A1
(43) Pub. Date: Jul. 6, 2023

(54) AAV Gene Therapy for Spastic Paraplegia

(71) Applicant: Genetic Cures for Kids Inc., Mons Queenslands (AU)

(72) Inventor: Neil R. Hackett, Santa Monica, CA (US)

(73) Assignee: Genetic Cures for Kids Inc., Mons Queenslands (AU)

(21) Appl. No.: 18/147,051

(22) Filed: Dec. 28, 2022

Related U.S. Application Data
(60) Provisional application No. 63/294,757, filed on Dec. 29, 2021.

(51) Int. Cl. A61K 48/00; A61K 9/00; A61P 21/00

(52) U.S. Cl. CPC A61K 48/0025; A61K 9/0019; A61P 21/00

(57) ABSTRACT

The present disclosure relates to AAV gene therapy vectors, AAV replicons, and pharmaceutical compositions for delivering a human CYP2U1 gene to a subject for treating hereditary spastic paraplegias, especially SPG56. In addition, methods of treatment and gene transfer are also provided as well as minimally invasive biomarkers for monitoring disease progression and other uses.

### CROSS REFERENCE TO RELATED APPLICATION

This application claims the benefit of provisional applications U.S. Ser. No. 63/294,757, filed Dec. 29, 2021 which is incorporated herein by reference in its entirety.

### SEQUENCE LISTING

The instant application contains an electronic Sequence Listing which has been submitted electronically in XML, format and is hereby incorporated by reference in its entirety. Said XML copy, created on Dec. 28, 2022, is named 9080_0101-us2.xml and is 16,533 bytes in size.

### FIELD OF THE DISCLOSURE

The present disclosure relates to AAV gene therapy vectors, AAV replicons, and pharmaceutical compositions for delivering a human CYP2U1 gene to a subject for treating hereditary spastic paraplegias, especially SPG56. In addition, methods of treatment and gene transfer are also provided as well as minimally invasive biomarkers for monitoring disease progression and other uses.

### BACKGROUND

Hereditary spastic paraplegias represent a series of genetic diseases with spasticity of the limbs and various other symptoms such as seizures, intellectual disability and poor fine motor control (Fink 2013). There are many types with diverse biochemical and genetic features but overlapping symptoms. Spastic paraplegia type 56 (SPG56) is a genetic disease arising from mutations that impair metabolism of lipids. The clinical syndrome is marked by spasticity beginning in the legs and generally presents at less than 2 years of age after a period of normal development. Additional symptoms vary and include upper limb spasticity, cognitive impairment, visual defects and brain calcification detected by CT scan. Disease progression is quite slow with most subjects reaching adulthood. No specific cures exist but intensive physical and occupation therapy are indicated. Recently, folate supplements have been suggested as helpful (Pujol 2021).

SPG56 is linked to mutations in the gene CYP2U1 which impairs the function of cytochrome P450 type 2U1. Cytochrome P450s are a superfamily of enzymes that use heme cofactors in hydroxylation reactions of steroids, lipids, fatty acids and many other molecules and in drug metabolism. P450s come in many forms with different substrate specificity (Tornio 2018; Waring 2020; Guengerich 2016). The CYP2U1 gene encodes an enzyme that hydroxylates arachidonic acid, a cellular signaling molecule, to create several bioactive derivatives, notably 19- and 20-hydroxyeicosatetraenoic acids (19- and 20-HETE, respectively). The connection between this biochemical pathway and motor neuron defects is not established but may be secondary to mitochondrial function. Based on fusions to GFP reporter, the CYP2U1 protein localizes to endoplasmic reticulum and mitochondria. Moreover, SPG56 cell lines have shown evidence of morphological and functional defects in mitochondria cell lines (Tesson 2016).

Case reports of SPG56 subjects have also established a recessive mode of inheritance with many nonsense, missense and frameshift mutations identified (Sharawat 2021; Kariminejad 2016; Bibi 2020; Citterio 2014; Masciullo 2016). The incidence of SPG56 is low and a recent exhaustive compilation listed 32 cases worldwide. Specific mutations were found in some populations suggesting a founder effect (Pujol 2021). One study expressed eight pathogenic variants of CYP2U1 in HEK cells (variants G115S, D316V, E380G, R3841, C262R, R488W, R390*, and C490Y) and concluded that missense variants in CYP2U1 were functionally inactive because of a loss of proper heme binding or destabilization of the protein structure (Durand 2018). The genetics clearly draws a line from the absence of CYP2U1 enzymatic activity to the resultant phenotype.

Because spastic paraplegias are recognized as neurological diseases which affect brain function and motor control, ideal therapeutic approaches to treatment would access the brain and motor neurons. Given that SPG56 arises from the absence of CYP2U1 expression due to genetic defects, gene therapy to deliver a good copy of the gene may be therapeutic. While a gene delivery system to all CYP2U1 deficient cells and that could mimic the endogenous expression pattern of CYP2U1 would be desirable, most known gene delivery systems are selective in which cell they can access. Accordingly, gene therapy vectors to deliver CYP2U1 to the brain and central nervous system are being developed.

### SUMMARY OF THE DISCLOSURE

The present disclosure relates to methods and gene therapy vectors for treating spastic paraplegias, and more particularly for treating SPG56, and diseases associated with defects in cytochrome P450 proteins, particularly CYP2U1. As more fully set forth below, the various embodiments and features described herein may be used independently of, or in combination, with each other, in all appropriate permutations.

Accordingly, in one aspect, the gene therapy (aka gene transfer) vectors of the disclosure are AAV vectors comprise an AAV replicon which comprises, in 5′ to 3′ direction, (i) a first AAV inverted terminal repeat (ITR), (ii) a promoter operably linked to a CYP2U1 open reading frame, (iii) a CYP2U1 open reading frame (SEQ ID NO:1), (iv) a polyadenylation (pA) signal operably linked to the CYP2U1 open reading frame, and (v) a second AAV ITR. In an embodiment, the second ITR is the inverse complement of the first ITR. In other embodiments, the ITRs can be the flip and flop configuration or any other configuration that produce infectious AAV vectors.

In an embodiment, the ITRs are from AAV serotype 2 or a neurotropic AAV serotype. In an embodiment, the promoter for controlling CYP2U1 gene expression is a human CYP2U1 promoter, a human EF1a promoter or a human SYN1 promoter. In an embodiment, the CYP2U1 open reading frame encodes an CYP2U1 protein having an amino acid sequence of FIG. 1 (SEQ ID NO: 1) or that of any other isoform of CYP2U1. In embodiments, the CYP2U1 open reading frame can encode a mutant CYP2U1protein that is therapeutically active. In an embodiment, the pA signal is a human growth hormone pA signal. In some embodiments the AAV replicon comprises one of the nucleic acid sequences in FIGS. 3A-C(SEQ ID NOS: 2-4, respectively).

In embodiments, suitable for non-human mammals, the promoters and CYP2U1 open reading frames can be from the non-human mammal, e.g., the promoter can be a murine CYP2U1 promoter, a murine EF1a promoter or a murine SYN1 used with a murine CYP2U1 protein, etc. Similarly, the pA signal can be a murine growth hormone pA signal when the AAV vector is for conducting gene therapy in a mouse,

In accordance with the disclosure, the replicon is present in a plasmid used to produce the AAV vectors of the disclosure.

In another aspect, the disclosure relates to recombinant AAV (rAAV) comprising AAV capsid proteins or AAV pseudocapsid proteins and an AAV replicon of the disclosure packaged therein. In an embodiment, the capsids are from AAV serotype 9 or a neurotropic AAV serotype.

A further aspect provides a pharmaceutical composition comprising an rAAV of the disclosure and a pharmaceutically-acceptable carrier.

Further aspects of the disclosure embrace methods for treating a spastic paraplegia, especially SPG56, or ameliorating one or more symptoms of a spastic paraplegia which comprises administering a pharmaceutical composition of the disclosure to a subject in an amount and for a time sufficient to treat the disease or to ameliorate one or more symptoms of the disease in the subject. In a preferred embodiment the spastic paraplegia is SPG56. In some embodiments, the subject is a rodent or a non-human primate. In some embodiment the subject is a human, including children, teenagers and adults. In embodiments, the composition is administered to the cerebrospinal fluid by lumbar puncture (intrathecal; IT) or injection into the cisterna magna (ICM) or cerebral ventricles (ICV).

A further aspect provides a method of gene transfer for treating a spastic paraplegia, preferably SPG56, or ameliorating one or more symptoms of spastic paraplegia, preferably SPG56, which comprises administering an rAAV of the disclosure to a mammal in an amount and for a time sufficient to treat or ameliorate the one or more symptoms of SPG56 in said mammal. In some embodiments, the mammal is a human, a rodent or a non-human primate. In some embodiment the mammal is a human, including children, teenagers and adults. In embodiments, the composition is administered by lumbar puncture, ICM, or ICV.

Additional aspects described herein relate to the use of biomarkers to determine efficacy of a therapeutic agent, to monitor efficacy during treatment and in treatments of spastic paraplegia and diseases associated with a cytochrome P450 defect. It has been discovered and first demonstrated herein that in a mouse model of spastic paraplegia, homozygous CYP2U1 knockout mice treated with an rAAV of the disclosure had decreased serum levels of Coenzyme Q8 and Coenzyme Q9 relative to untreated knockout mice, thus providing a simple, non-invasive biomarker for assessing treatment, monitoring treatment and for finding potential therapeutic agents.

Accordingly for the biomarker uses embrace, in an embodiment, methods for determining efficacy of a therapeutic agent in treating a spastic paraplegia or a disease associated with a cytochrome P450 defect which comprise
(a) administering a candidate therapeutic agent to a subject; and
(b) monitoring a level of one or more Coenzyme Q isoforms in a bodily fluid of said subject relative to the level in the subject prior to administering the agent, wherein a decrease in the level of one or more Coenzyme Q isoforms relative to baseline indicates the agent is effective for treating spastic paraplegia or the cytochrome P450-associated disease.

In another embodiment for these uses of biomarkers, the disclosure provides methods to monitor therapeutic efficacy in treating or ameliorating a spastic paraplegia or a disease associated with a cytochrome P450 defect which comprise
(a) measuring a baseline level of one or more Coenzyme Q isoforms in a bodily fluid of a subject having or suspected of having a spastic paraplegia or a disease associated with a cytochrome P450 defect;
(b) administering a dosage of a therapeutic agent to the subject;
(c) measuring a post-administration level of one or more Coenzyme Q isoforms in a bodily fluid of the subject, wherein a decrease in one or more Coenzyme Q isoforms relative to baseline indicates that the therapeutic agent is efficacious against spastic paraplegia or the disease associated with a cytochrome P450 defect; and
(d) continuing administering the therapeutic agent as needed to maintain decreased levels of one or more Coenzyme Q isoforms relative to baseline levels.

In an further embodiment of these uses of biomarkers, the disclosure provides methods for treating or ameliorating spastic paraplegia or a disease associated with cytochrome P450 defects which comprise
(a) measuring a baseline level of one or more Coenzyme Q isoforms in a bodily fluid of a subject having a spastic paraplegia or a disease associated with a cytochrome P450 defect;
(b) administering a therapeutic agent to said subject; and
(c) measuring a post-administration level of one or more Coenzyme Q isoforms in a bodily fluid of the said subject, wherein a decrease in one or more Coenzyme Q isoforms relative to baseline indicates said spastic paraplegia or said disease associated with a cytochrome P450 defect is responding to said therapeutic agent.

In any of the foregoing embodiments, the spastic paraplegia can be a hereditary spastic paraplegia or sporadic spastic paraplegia. Examples of spastic paraplegia include, but are not limited to, SPG3A, SPG4, SPG5A, SPG7, SPG10, SPG11, SPG13, SPG19, SPG26, SPG28, SPG31, SPG35, SPG39, SPG42, SPG46, SPG56, SPG66, SPG67, SPG81, SPG, 81, SPG82, SPG84 and SPG86. In a preferred embodiment, the spastic paraplegia is SPG56.

In any of the foregoing embodiments, the cytochrome P450-associated disease is a disease associated with CYP2U1.

In any of the foregoing embodiments, levels of one or more Coenzyme Q isoforms are measured one or more times (e.g., at baseline and at various times after administering the therapeutic agent). The one or more Coenzyme Q isoforms that re measure are preferably Coenzyme Q8, Coenzyme Q9 or both.

In any of the foregoing embodiments, the bodily fluid is peripheral blood, serum, plasma, ascites, urine, saliva or cerebrospinal fluid (CSF), and preferably is serum.

In any of the foregoing embodiments, the therapeutic agent can be a recombinant AAV comprising an AAV replicon comprising a promoter operably linked to a functional protein coding region (i.e., to provide a good copy of the defective gene) associated with the spastic paraplegia to be treated or associated with the disease associated with the cytochrome P450 defect to be treated.

In embodiments relating to methods of determining efficacy of a therapeutic agent in treating a spastic paraplegia or a disease associated with a cytochrome P450 defect, any target therapeutic agent can be assessed or evaluated for efficacy. For example, small molecule therapeutics can be administered. Likewise, the therapeutic agent can be any gene therapy vector which can provide a good copy of the defective gene for a particular spastic paraplegia. In preferred embodiments, those gene therapy vectors are rAAV since these are neurotropic vectors. The defective genes in spastic paraplegias (SPG diseases) are well known to those of skill in the art. A list of SPG diseases can be found, for example, on the website of the Neuromuscular Disease Center, Washington University, St. Louis, Mo., at https://neuromuscular.wustl.edu/spinal/fsp.html.

### BRIEF DESCRIPTION OF THE DRAWINGS

FIG. 1 provides the amino acid sequence of the CYP2U1 isoform X1 protein (SEQ ID NO: 1), which has 562 amino acids and a mass of 63,794 Daltons.

FIGS. 2A-C depict schematic diagrams of the AAV SPG56 vectors for (A) AAV-pEF1a-hCYP2U1, (B) AAV-pSYN1-hCYP2U1, and (C) AAV-pCYP2U1-hCYP2U1.

FIGS. 3A-C provide the nucleic acid sequence of the replicon portion of the AAV SPG56 vectors for (A) AAV-pEF1a-hCYP2U1, (B) AAV-pSYN1-hCYP2U1, and (C) AAV-pCYP2U1-hCYP2U1, which are provided in the sequence listing as SEQ ID NOS: 2-4, respectively.

FIG. 4 shows the effect of neonatal intra-cerebroventricular gene therapy on muscle tension as a function of stimulation frequency, assessed at 8 weeks of age. Experiments were conducted as described in Example 4. Homozygous SP56 knockout mice received no vector (•), AAV-pEF1a-mCYP2U1 (▪), or AAV-pSYN1-mCYP2U1 (▴). Control wildtype mice were untreated (▾).

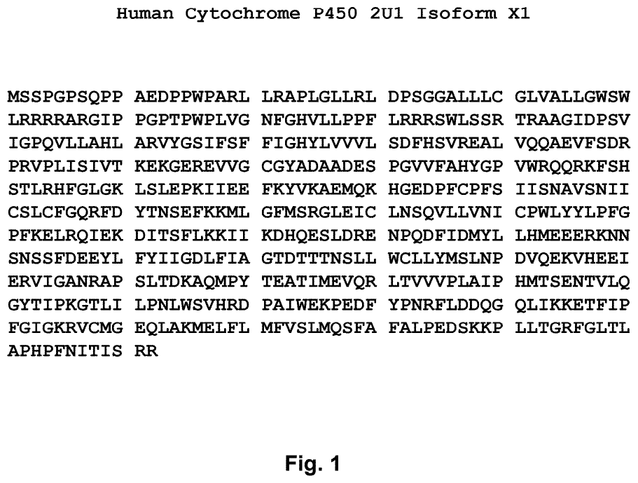

### DETAILED DESCRIPTION OF THE INVENTION

### Definitions

In order that the present invention may be more readily understood, certain terms are defined below. Additional definitions may be found within the detailed description of the invention.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., cows, pigs), companion animals (e.g., dogs, cats) and rodents (e.g., mice and rats).

The term “non-human mammal” means a mammal which is not a human and includes, but is not limited to, a mouse, rat, rabbit, pig, cow, sheep, goat, dog, non-human primate, or other non-human mammals typically used in research. As used herein, “mammals” includes the foregoing non-human mammals and humans.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment and is not necessarily meant to imply complete cessation of the relevant disease, disorder or condition.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition or preventing or delaying the development of symptoms associated with the condition.

As used herein, an “effective amount,” “therapeutically-effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

As used herein, the term “nucleic acid molecule” is intended to include DNA molecules, RNA molecules (e.g., mRNA, shRNA, siRNA, microRNA), analogs of the DNA or RNA generated using nucleotide analogs, and derivatives, fragments and homologs thereof. The nucleic acid molecules of the invention may be single-, double-, or triple-stranded. A nucleic acid molecule of the present invention may be isolated using sequence information provided herein and well known molecular biological techniques (e.g., as described in Sambrook et al., Eds., MOLECULAR CLONING: A LABORATORY MANUAL 2ND ED., Cold Spring Harbor Laboratory Press, Cold Spring Harbor, N.Y., 1989; and Ausubel, et al., Eds., CURRENT PROTOCOLS IN MOLECULAR BIOLOGY, John Wiley & Sons, New York, N.Y., 1993).

A “vector” refers to a macromolecule or association of macromolecules that comprises or associates with a polynucleotide, and which can be used to mediate delivery of the polynucleotide to a cell, either in vitro or in vivo, illustrative vectors include, for example, plasmids, viral vectors, liposomes and other gene delivery vehicles, such as viruses. The polynucleotide to be delivered, sometimes referred to as a “target polynucleotide” or “transgene,” may comprise a coding sequence of interest in gene therapy (such as a gene encoding a protein of therapeutic interest). Vector thus includes a biological entity, such as an AAV or other virus, used for the delivery of genes into an organism or introduction of foreign genes into cells.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels). e.g., by ELISA. How cytometry and Western blot, measurement of DNA and RNA by heterologous hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” or “gene transfer” refers to the introduction of an exogenous polynucleotide into a cell for gene therapy, and may encompass targeting, binding, uptake, transport, localization, replicon integration and expression.

“Gene therapy” refers to the introduction of an exogenous polynucleotide into a cell which may encompass targeting, binding, uptake, transport, localization and replicon integration, but is distinct from and does not imply subsequent expression of the gene.

“Gene expression” refers to the process of gene transcription, translation, and post-translational modification.

An “infectious” virus or viral particle is one that comprises a polynucleotide component which it is capable of delivering into a cell for which the viral species e trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components, if present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments are envisioned. Thus for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence (TRS) or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one aide of the terminator from continuing through to the other side of the terminator). The degree to which transcription is disrupted is typically a function of the base sequence and/or the length of the terminator sequence. In particular, as is well known in numerous molecular biological systems, particular DMA sequences, generally referred to as transcriptional termination sequences' are specific sequences that tend to disrupt read-through transcription by RNA polymerase, presumably by causing the RNA polymerase molecule to stop and/or disengage from the DMA being transcribed. Typical example of such sequence-specific terminators include polyadenylation (“polyA”) sequences, e.g., SV40 polyA. In addition to or in place of such sequence-specific terminators, insertions of relatively long DMA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators) and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures.” “packaging cell line” and other such terms denote higher eukaryotic cells, such as mammalian cells including human cells, useful in the present disclosure, e.g., to produce recombinant virus or recombinant fusion polypeptide. These cells Include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector comprising a region which encodes a gene product of interest and is used for effecting the expression of the gene product in an intended target cell. An expression vector also comprises control elements operatively linked to the encoding region to facilitate expression of the protein in the target. The combination of control elements and a gene or genes to which they are operably linked for egression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a afferent gene.

“Transformed” or “transgenic” is used herein to include any host cell or cell line, which has been altered or augmented by the presence of at least one recombinant DNA sequence. The host cells are typically produced by transfection with a DNA sequence in a plasmid expression vector, as an isolated linear DNA sequence, or infection with a recombinant viral vector.

### AAV Vectors

The present disclosure provides AAV vectors for use in gene therapy for treating hereditary spastic paraplegias and more particularly for treating spastic paraplegia 56 (SPG56) and other diseases associated with mutations in CYP2U1. The CYP2U1 gene is expressed at a low level in multiple cell types and the tropism of AAV allows expression in many cell types. In treating SPG56, obtaining a similar level of expression as the endogenous CYP2U1 gene may be desired. Thus, AAV vectors can be designed to express a low level of CYP2U1 and be delivered in ways that the transduce as many cells as possible. For treating the neurological manifestations of SPG56, AAV vectors with neuro tropism can be delivered to the brain by or by direct injection into the cerebrospinal fluid via the cistern magna which affords direct access to the neurons of the CNS.

In accordance herewith, the AAV vector of the disclosure is for delivery of an AAV replicon comprising, in 5′ to 3′ direction, a first AAV inverted terminal repeat (ITR), a promoter operably linked to an CYP2U1 open reading frame, a polyadenylation (pA) signal, and a second AAV ITR.

AAV vectors have many applications in gene therapy for many reasons, including their tropism for specific cell types, their ability to infect both dividing and non-dividing cells, and their ability for genomic integration.

AAV comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids have approximately 96% of the parental genome deleted, such that only the terminal repeats (ITRs), which contain recognition signals for DNA replication and packaging, remain.

AAV vectors of the disclosure can be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316-327 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, B. J., Hum. Gene Ther., 16: 541-550 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71: 6823-33 (1997); Srivastava et al., J. Virol., 45: 555-64 (1983); Chiorini et al., J. Virol., 73: 1309-1319 (1999); Rutledge et al., J. Virol., 72: 309-319 (1998); and Wu et al., J. Virol., 74: 8635-47 (2000)).

Generally, the capsid proteins, which determine the cellular tropism of the AAV particle, and related capsid protein-encoding sequences (cap), are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1): 1-2 (2006), Gao et al., J. Virol., 78: 6381-6388 (2004), Gao et al., Proc. Natl. Acad. Sci. USA, 99: 11854-11859 (2002), De et al., Mol. Ther., 13: 67-76 (2006), and Gao et al., Mol. Ther., 13: 77-87 (2006).

In embodiments, the transgene in the AAV replicon has the cDNA for a human or other mammalian CYP2U1 gene operably linked to a promoter capable of controlling its expression at therapeutic levels. The exact therapeutic level can be determined by those of skill in the art. Expression of the transgene from gene therapy vectors can be driven by any promoter, including strong promoters such as cytomegalovirus (CMV) and chicken beta actin (CBA) that express in all cell types (Gray 2011). In preferred embodiments, weaker promoters are used including elongation factor 1A (Ef1a), or phosphoglycerol kinase (PGK), or a native CYP2U1 promoter. Since the CYP2U1 phenotype results in a neurological phenotype, neuron specific promoters such as methyl CpG-binding protein 2 (MEP229 MEP545), synapsin (SYN1), somatostatin (SST) can be used, potentially reducing toxicity due to ectopic expression of transgene, especially in liver (Gadalla 2017; Sinnett 2017; Peviani 2012). For example, expression in GABAnergic neurons can also be achieved using cell-specific promoters (Peviani 2012; Egashira 2018).

In an embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). In some embodiments, the AAV vector is generated using an AAV that infects a non-human primate pseudotyped with an AAV that infects humans. Examples of such pseudotyped AAV vectors are disclosed in, e.g., Cearley et al., Molecular Therapy, 13: 528-537 (2006). In one embodiment, an AAV vector can be generated which comprises a capsid protein from an AAV that infects rhesus macaques pseudotyped with AAV2 inverted terminal repeats (ITRs). In another embodiment, the AAV vector comprises a capsid protein from AAV9 in which the genome derived from AAV2 is pseudotyped into AAV9 capsid. In another embodiment, AAV10 (also referred to as “AAVrh.10”), which infects rhesus macaques, is pseudotyped with AAV2 ITRs (see, e.g., Watanabe et al., Gene Ther., 17(8): 1042-1051 (2010); and Mao et al., Hum. Gene Therapy, 22: 1525-1535 (2011)).

A common AAV vector production strategy is triple transfection method, which involves co-transfecting the packaging cell line (usually HEK293 T) with the recombinant AAV plasmid containing the gene of interest (GOI), a plasmid containing the essential rep and cap genes, and a third adenovirus-derived helper plasmid supplying genes needed for replication. For large-scale and preclinical AAV packaging services, the AAV particles are purified using IDX gradient ultracentrifugation to remove impurities and empty capsids. In general, methods of producing and purifying AAV vectors using two plasmid and three plasmid systems are known in the art and any such methods can be used to produce the AAV vectors disclosed herein, see, e.g., U.S. Pat. Nos. 6,503,888; 6,632,670; 8,007,780; 8,642,341; 9,051,542; 10,017,746; 10,087,224; 10,093,947; and 10,982,228.

In some embodiments, virions containing a recombinant AAV vector are prepared based on procedures described by Kantor et al. (Advances in Genetics, vol. 87, 2014, Chapter 2, “Clinical Applications Involving CNS Gene Transfer”); Kaplitt et al. (Lancet 369: 2097-105, 2007); Worgall et al. (Human Gene Therapy 19:463-474 (2008); Leone et al., Sci. Transl Med 4: 165ra163 (2012). In an embodiment, the AAV vector suitable for use in the present invention is produced according to the methods described in U.S. Pat. No. 6,342,390. In an alternate embodiment, the AAV vector suitable for use in the present disclosure is produced according to the methods described in U.S. Pat. No. 6,821,511.

Packaging cell lines include 293 cells which are human embryonic kidney cells modified to contain a small fragment of human adenovirus genome which includes the adenoviral Ela and E1b genes. Another useful packaging cell line is the 293T cell line which contains the SV40 large T antigen gene Both 293 and 293T cells are readily transfected and efficiently package replication deficient AAV vectors given the other adenovirus helper functions (E2a, E4) in the first helper plasmid and AAV replications and capsid functions in the second helper plasmid.

### Methods of Treatment

The present disclosure provides AAV gene therapy vectors for treating a hereditary spastic paraplegia associated with defects in the CYP2U1 gene and particularly SPG56. Particular embodiments included methods for treating or ameliorating one or more symptoms of SPG56 which comprises administering an AAV vector of the disclosure or a pharmaceutical composition of the disclosure to a subject in an amount and for a time sufficient to treat or ameliorate the one or more symptoms of SPG56 in the subject.

In some embodiments the AAV vector or composition is administered into the cerebrospinal fluid, into the brain, into the cisterna magna or into the circulatory system using delivery methods known in the art. In some embodiments, delivery into the cerebrospinal fluid is done by lumbar puncture. In some embodiments, delivery is via an intracerebroventricular (ICV) or intravenous (IV) route. In some embodiments, delivery is intra cisterna magna.

In some embodiments, the subject is a rodent or a non-human primate. In some embodiments the subject is a human. In some embodiments, the subject has been diagnosed with a known SPG56 mutation but has not yet exhibited disease symptoms. Many SPG56 mutations in CYP2U1 that lead to disease are known and diagnostics therefor (such as sequencing) are available to those of skill in the art (Sharawat 2021; Kariminejad 2016; Bibi 2020; Citterio 2014; Masciullo 2016; Pujol 2021). Examples of such mutations include the variants G115S, D316V, E380G, R3841, C262R, R488W, R390*, and C490Y (Durand 2018).

In accordance with the method for treating SPG56, and other spastic paraplegias, the symptoms to be evaluated include, but are not limited to, neuromuscular status, occurrence of seizures, intellectual ability and development, behavior, gross motor ability, fine motor ability, spatial recognition and muscular strength. The Spastic Paraplegia Rating Scale (SPRS) provides a reliable and accepted method to evaluate symptoms during treatment (Schüle, 2006).

Additional other aspects of the SPG56 that can be evaluated, and which reflect a change of symptoms, include physiological parameters such as lipid analysis and changes in ubiquinols and ubiquinones, such as ubiquinol 9, ubiquinone 9, ubiquinol 10 and ubiquinone 10. For non-human subjects, efficacy of treatment can include gross examination of tissues and histology

The present disclosure also contemplates a method of gene transfer for treating or ameliorating one or more symptoms of SPG56 which comprises administering an AAV vector or pharmaceutical composition of the disclosure to a mammal in an amount and for a time sufficient to treat or ameliorate the one or more symptoms of SPG56 in said mammal. In some embodiments the AAV vector or composition is administered by intracerebroventricular or intravenous routes.

In some embodiments, the mammal is a rodent or a non-human primate. In some embodiments the mammal is a human.

The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. See, e.g., Remington: The Science and Practice of Pharmacy 21st Ed., Univ. of Sciences in Philadelphia (USIP), Lippincott Williams & Wilkins, Philadelphia, Pa., 2005.

### Pharmaceutical Compositions, Administration and Dosing

The present invention further provides pharmaceutical compositions comprising a AAV vector of the disclosure, together with a pharmaceutically acceptable carrier, excipient or vehicle.

Accordingly, the present invention further provides a pharmaceutical composition comprising an AAV vector of the disclosure. Certain embodiments of the pharmaceutical compositions of the invention are described in further detail below.

An AAV vector of the disclosure may be formulated as pharmaceutical compositions prepared for storage or administration, which typically comprise a therapeutically effective amount of the vector in a pharmaceutically acceptable carrier.

The therapeutically-effective amount of the AAV vector of the disclosure will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by animal studies and confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. pH buffering agents may be phosphate, citrate, acetate, tris/hydroxymethyl)aminomethane (TRIS), N-Tri s(hydroxymethyl)methyl aminopropanesulphonic acid (TAPS), ammonium bicarbonate, diethanolamine, histidine, which is a preferred buffer, arginine, lysine, or acetate or mixtures thereof. The term further encompasses any agents listed in the US Pharmacopeia for use in animals, including humans.

The term “pharmaceutically-acceptable salt” refers to the salt of the compounds. As used herein a pharmaceutically-acceptable salt retains qualitatively a desired biological activity of the parent compound without imparting any undesired effects relative to the compound. Salts include pharmaceutically acceptable salts such as acid addition salts and basic salts. Acid addition salts include salts derived from nontoxic inorganic acids, such as hydrochloric, nitric, phosphorous, phosphoric, sulfuric, hydrobromic, hydroiodic and the like, or from nontoxic organic acids such as aliphatic mono- and di-carboxylic acids, phenyl-substituted alkanoic acids, hydroxy alkanoic acids, aromatic acids, aliphatic and aromatic sulfonic acids and the like. Examples of basic salts include salts where the cation is selected from alkali metals, such as sodium and potassium, alkaline earth metals such as calcium and magnesium, and ammonium ions ⁺N(R³)₃(R⁴), where R³ and R⁴ independently designate optionally substituted C₁₋₆-alkyl, optionally substituted C₂₋₆-alkenyl, optionally substituted aryl, or optionally substituted heteroaryl, and more specifically, the organic amines, such as N, N′-dibenzylethylenediamine, N-methylglucamine, chloroprocaine, choline, diethanolamine, ethylenediamine, procaine and the like. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration.

Pharmaceutically-acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for the compounds described herein.

An acceptable route of administration may refer to any administration pathway known in the art, including but not limited to aerosol, enteral, nasal, ophthalmic, oral, parenteral, rectal, vaginal, or transdermal (e.g., topical administration of a cream, gel or ointment, or by means of a transdermal patch). “Parenteral administration” is typically associated with injection at or in communication with the intended site of action, including infraorbital, intraarterial, intracapsular, intracardiac, intracerebroventricular, intradermal, intramuscular, intraperitoneal, intrapulmonary, intraspinal, intracisternal, intrathecal, intrauterine, intravenous, subarachnoid, subcapsular, subcutaneous, transmucosal, or transtracheal administration.

Pharmaceutical compositions of the disclosure may be administered alone or in combination with one or more other therapeutic or diagnostic agents. A combination therapy may include an AAV vector of the disclosure combined with at least one other therapeutic agent selected based on the particular patient, disease or condition to be treated. Examples of other such agents include, inter alia, an a psychoactive drug, anti-inflammatory or anti-proliferative agent, growth factors, cytokines, an analgesic, a therapeutically-active small molecule or polypeptide, a single chain antibody, a classical antibody or fragment thereof, or a nucleic acid molecule which modulates expression of one or more genes, one or more modifiers of signaling pathways and similar modulating therapeutics which may complement or otherwise be beneficial in a therapeutic or prophylactic treatment regimen.

As used herein, “pharmaceutically acceptable carrier” includes any and all physiologically acceptable, i.e., compatible, solvents, dispersion media, coatings, antimicrobial agents, isotonic and absorption delaying agents, and the like. In certain embodiments, the carrier is suitable for intravenous, intramuscular, subcutaneous, parenteral, spinal or epidermal administration (e.g., by injection or infusion). Depending on selected route of administration, the AAV vector may be coated in a material or materials intended to protect it from the action of acids and other natural inactivating conditions to which the AAV vector may encounter when administered to a subject by a particular route of administration.

A pharmaceutical composition of the invention also optionally includes a pharmaceutically acceptable antioxidant. Exemplary pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants.

Compositions of the disclosure may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

Exemplary pharmaceutically acceptable carriers include sterile aqueous solutions or dispersions and sterile powders for the extemporaneous preparation of sterile injectable solutions or dispersion. Such media and reagents for pharmaceutically active substances are known in the art. The pharmaceutical compositions of the disclosure may include any conventional media or agent unless any is incompatible with the AAV vectors of the disclosure. Supplementary active compounds may further be incorporated into the compositions.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g., sugars, polyalcohols such as mannitol, sorbitol, or sodium chloride may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating an AAV vector of the disclosure in the required amount in an appropriate solvent with one or a combination of ingredients described above, as required, followed by sterilization microfiltration. Dispersions may be prepared by incorporating the active compound into a sterile vehicle that contains dispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of an AAV vector of the disclosure is administered by, e.g., intravenous, intracisternal, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition of the present invention may also contain stabilizers, preservatives, buffers, antioxidants, or other additives well known to those of skill in the art.

The amount of active ingredient which can be combined with a carrier material to produce a single dosage form will vary depending on a variety of factors, including the subject being treated, and the particular mode of administration. In general, it will be an amount of the composition that produces an appropriate therapeutic effect under the particular circumstances. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens may be adjusted to provide the optimum desired response (e.g., a therapeutic response). For example, a single bolus may be administered, several divided doses may be administered over time, or the dose may be proportionally reduced or increased as indicated by the particular circumstances of the therapeutic situation, on a case by case basis. It is especially advantageous to formulate parenteral compositions in dosage unit forms for ease of administration and uniformity of dosage when administered to the subject or patient. As used herein, a dosage unit form refers to physically discrete units suitable as unitary dosages for the subjects to be treated; each unit containing a predetermined quantity of active compound calculated to produce a desired therapeutic effect in association with the required pharmaceutical carrier. The specification for the dosage unit forms depends on the specific characteristics of the active compound and the particular therapeutic effect(s) to be achieved, taking into consideration and the treatment and sensitivity of any individual patient.

For administration of an AAV vector, the dosage range will generally be from about 2×10¹⁰ to 5×10¹⁵ genome copies or 1×10¹¹ to 1×10¹⁵ of the host body weight. Exemplary dosages may be 3×10¹² genome copies/kg body weight, 1×10¹³ genome copies/kg body weight, 3×10¹³ genome copies/kg body weight, 1×10¹⁴ genome copies/kg body weight or 3×10¹⁴ genome copies/kg body weight or within the range of 1×10¹² to 3×10″ genome copies/kg. Dosages may be selected and readjusted as required to maximize therapeutic benefit for a particular subject.

AAV vectors may be administered one or more times. Intervals between single dosages can be, for example, yearly or longer, including 1 year, 2 years, 5 years, or 10 years.

In certain embodiments, two or more AAV vectors may be administered simultaneously or sequentially, in which case the dosage of each administered compound may be adjusted to fall within the ranges described herein.

Actual dosage levels of the AAV vector alone or in combination with one or more other active ingredients in the pharmaceutical compositions of the present invention may be varied so as to obtain an amount of the active ingredient which is effective to achieve the desired therapeutic response for a particular patient, composition, and mode of administration, without causing deleterious side effects to the subject or patient. A selected dosage level will depend upon a variety of factors, such as pharmacokinetic factors, including the activity of the particular AAV vector employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject or patient being treated, and similar factors well known in the medical arts.

Administration of a “therapeutically effective dosage” of an AAV vector of the disclosure may result in a decrease in severity of disease symptoms, an increase in frequency and duration of disease symptom-free periods, or a prevention or lessening of impairment or disability due to the disease affliction.

The AAV vector or composition of the present disclosure may be administered via one or more routes of administration, using one or more of a variety of methods known in the art. As will be appreciated by the skilled worker, the route and/or mode of administration will vary depending upon the desired results. Routes of administration for AAV vectors and compositions containing such vectors invention include, e.g., intracerebroventricular, intravenous, intraperitoneal, subcutaneous, spinal or other parenteral routes of administration, for example by injection or infusion. The phrase “parenteral administration” as used herein refers to modes of administration other than enteral and topical administration, usually by injection, and includes, without limitation, intravenous, intramuscular, intraarterial, intrathecal, intracapsular, intraorbital, intracardiac, intraperitoneal, subcuticular, intraarticular, subcapsular, subarachnoid, epidural and intracisternal magna injection and infusion.

As described elsewhere herein, an AAV vector may be prepared with carriers that will protect it against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compounds or compositions of the invention may be administered with one or more of a variety of medical devices known in the art. For example, in one embodiment, a therapeutic AAV vector composition of the disclosure may be administered with a needleless hypodermic injection device. Examples of well-known implants and modules useful in the present invention are in the art, including e.g., implantable micro-infusion pumps for controlled rate delivery; infusion pumps for delivery at a precise infusion rate; and injection catheters that direct the drug to specific body compartments. These and other such implants, delivery systems, and modules are known to those skilled in the art.

### Use of Coenzyme Q Isoforms as Biomarkers

In observing the positive impact of treatment in a knockout mouse model for SPG56 by AAV gene transfer to cerebrospinal fluid on disease progression, it was discovered that Coenzyme Q8 (CoQ8) and Coenzyme Q9 (CoQ9) could provide a minimally invasive, objective biomarker to assess the impact of treatment for spastic paraplegia, especially in human subjects. Accordingly, the disclosure relates the use of biomarkers to determine efficacy of a therapeutic agent, to monitor efficacy during treatment and in treatments of spastic paraplegia and diseases associated with a cytochrome P450 defect. As shown in Example 4, homozygous CYP2U1 knockout mice had significantly increased serum levels of CoQ8 and CoQ9 over wildtype mice—and that these levels were significantly reduced upon treatment of the knockout mice with an rAAV of the disclosure.

Accordingly, the Coenzyme Q isoforms can be used as biomarkers in the methods described herein.

In an embodiment, the disclosure provides methods for determining efficacy of a therapeutic agent in treating a spastic paraplegia or a disease associated with a cytochrome P450 defect which comprise
(a) administering a candidate therapeutic agent to a subject; and
(b) monitoring a level of one or more Coenzyme Q isoforms in a bodily fluid of said subject relative to the level in the subject prior to administering the agent, wherein a decrease in the level of one or more Coenzyme Q isoforms relative to baseline indicates the agent is effective for treating spastic paraplegia or the cytochrome P450-associated disease.

In an embodiment of the foregoing method, the subject can be in need of treatment. In an embodiment of the foregoing method, the subject can be a knockout mouse model for the spastic paraplegia. In some embodiments the subject is a CPY2U1 knockout mouse.

In another embodiment for use of these biomarkers, the disclosure provides methods to monitor therapeutic efficacy in treating or ameliorating a spastic paraplegia or a disease associated with a cytochrome P450 defect which comprise
(a) measuring a baseline level of one or more Coenzyme Q isoforms in a bodily fluid of a subject having or suspected of having a spastic paraplegia or a disease associated with a cytochrome P450 defect;
(b) administering a dosage of a therapeutic agent to the subject;
(c) measuring a post-administration level of one or more Coenzyme Q isoforms in a bodily fluid of the subject, wherein a decrease in one or more Coenzyme Q isoforms relative to baseline indicates that the therapeutic agent is efficacious against spastic paraplegia or the disease associated with a cytochrome P450 defect; and
(d) continuing administering the therapeutic agent as needed to maintain decreased levels of one or more Coenzyme Q isoforms relative to baseline levels.

In a further embodiment of these uses of biomarkers, the disclosure provides methods for treating or ameliorating spastic paraplegia or a disease associated with cytochrome P450 defects which comprise
(a) measuring a baseline level of one or more Coenzyme Q isoforms in a bodily fluid of a subject having a spastic paraplegia or a disease associated with a cytochrome P450 defect;
(b) administering a therapeutic agent to said subject; and
(c) measuring a post-administration level of one or more Coenzyme Q isoforms in a bodily fluid of the said subject, wherein a decrease in one or more Coenzyme Q isoforms relative to baseline indicates said spastic paraplegia or said disease associated with a cytochrome P450 defect is responding to said therapeutic agent.

In any of the foregoing embodiments, the spastic paraplegia (SPG) can be a hereditary spastic paraplegia or sporadic spastic paraplegia. Examples of spastic paraplegia include, but are not limited to, SPG3A, SPG4, SPG5A, SPG7, SPG10, SPG11, SPG13, SPG19, SPG26, SPG28, SPG31, SPG35, SPG39, SPG42, SPG46, SPG56, SPG66, SPG67, SPG81, SPG, 81, SPG82, SPG84 and SPG86. In a preferred embodiment, the spastic paraplegia is SPG56. A comprehensive list of SPG diseases can be found, for example, on the website of the Neuromuscular Disease Center, Washington University, St. Louis, Mo. (https://neuromuscular.wustl.edu/spinal/fsp.html).

In any of the foregoing embodiments, the cytochrome P450-associated disease is a disease associated with CYP2U1.

In any of the foregoing embodiments, levels of one or more Coenzyme Q isoforms are measured one or more times (e.g., at baseline and at various times after administering the therapeutic agent). The one or more Coenzyme Q isoforms that re measure are preferably Coenzyme Q8, Coenzyme Q9 or both. Methods for measuring Coenzyme Q levels are known to those of skill in the art and are described for example in Pujol 2021.

In any of the foregoing embodiments, the bodily fluid is peripheral blood, serum, plasma, ascites, urine, saliva or cerebrospinal fluid (CSF), and preferably is serum.

In any of the foregoing embodiments, the therapeutic agent can be a recombinant AAV comprising an AAV replicon comprising a promoter operably linked to a functional protein coding region (i.e., to provide a good copy of the defective gene) associated with the spastic paraplegia to be treated or associated with the disease associated with the cytochrome P450 defect to be treated. In an embodiment, the rAAV would encode CYP2U1 and the SPG for treatment or monitoring is SPG56.

In embodiments relating to methods of determining efficacy of a therapeutic agent in treating a spastic paraplegia or a disease associated with a cytochrome P450 defect, any target therapeutic agent can be assessed or evaluated for efficacy. For example, small molecule therapeutics or cell-based therapeutics can be administered and evaluated for activity. Likewise, the therapeutic agent can be any gene therapy vector which can provide a good copy of the defective gene for a particular spastic paraplegia. In preferred embodiments, those gene therapy vectors are rAAV since these are neurotropic vectors. The defective genes in spastic paraplegias (SPG diseases) are well known to those of skill in the art. A list of SPG diseases can be found, for example, on the website of the Neuromuscular Disease Center, Washington University, St. Louis, Mo., at https://neuromuscular.wustl.edu/spinal/fsp.html.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety.

### Examples

The examples presented herein represent certain embodiments of the present disclosure. However, it is to be understood that these examples are for illustration purposes only and do not intend, nor should any be construed, to be wholly definitive as to conditions and scope of this disclosure. The examples were carried out using standard techniques, which are well known and routine to those of skill in the art, except where otherwise described in detail.

### Example 1. Vector Construction and AAV Particle Production

Three AAV gene therapy vectors were constructed using a three-plasmid system with an AAV serotype 2 replicon-transgene plasmid and two helper plasmids. The three CYP2U1 transgene constructs and associated promoters described below were made by total synthesis and cloned into plasmids with the AAV terminal repeats in reverse complement configuration.

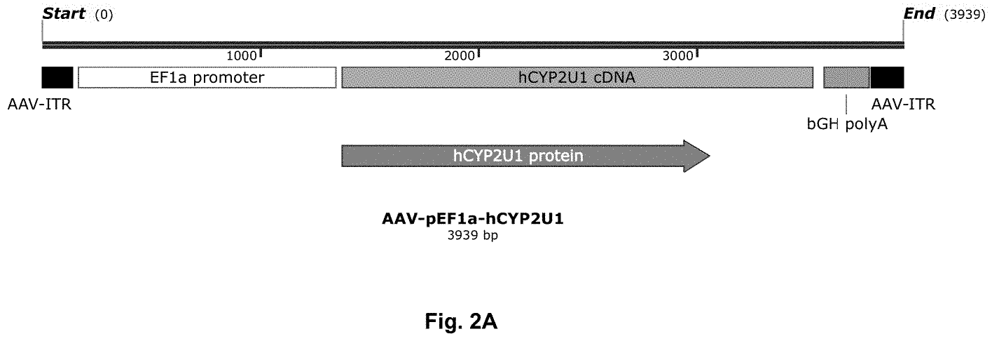

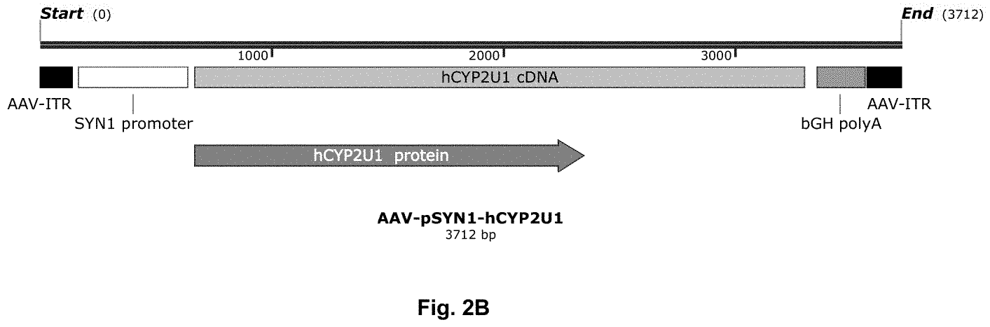

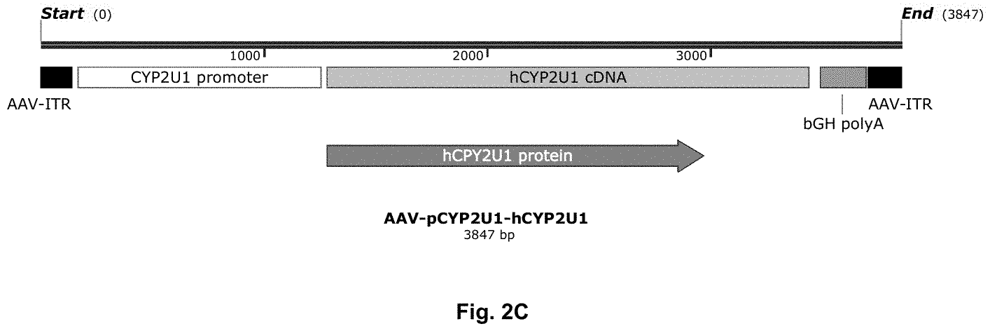

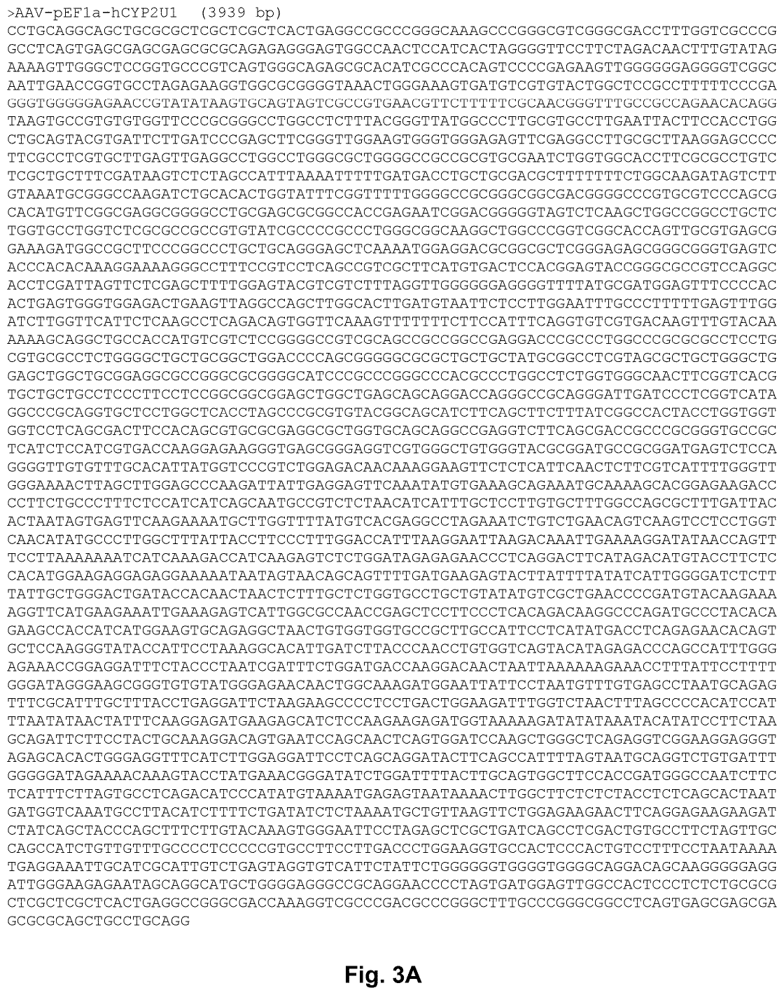

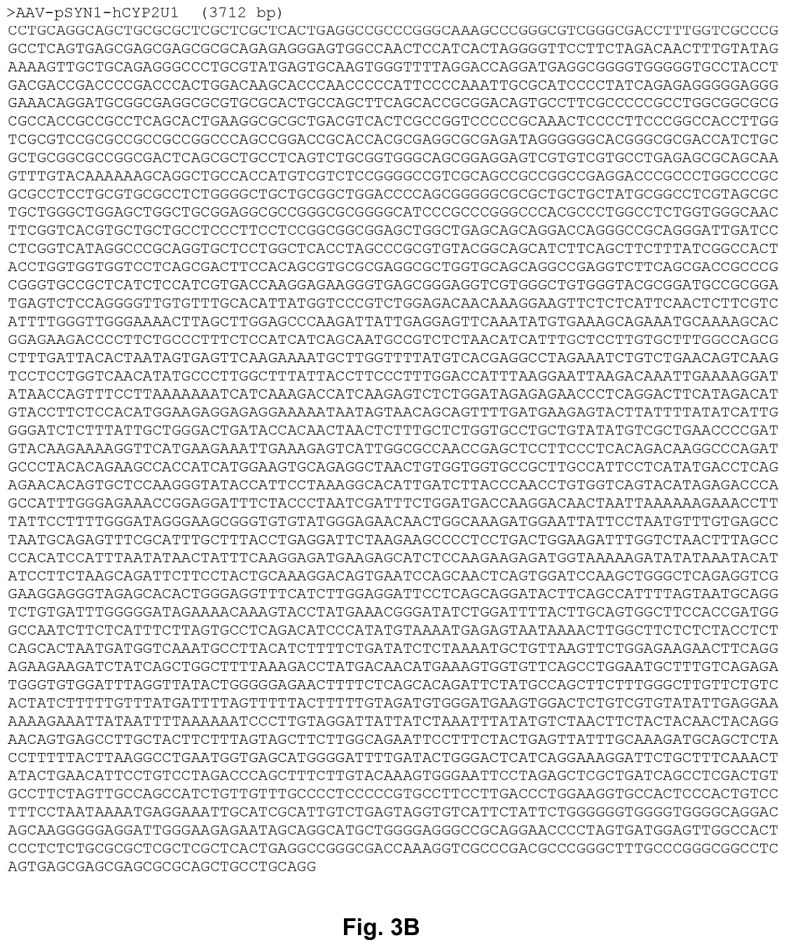

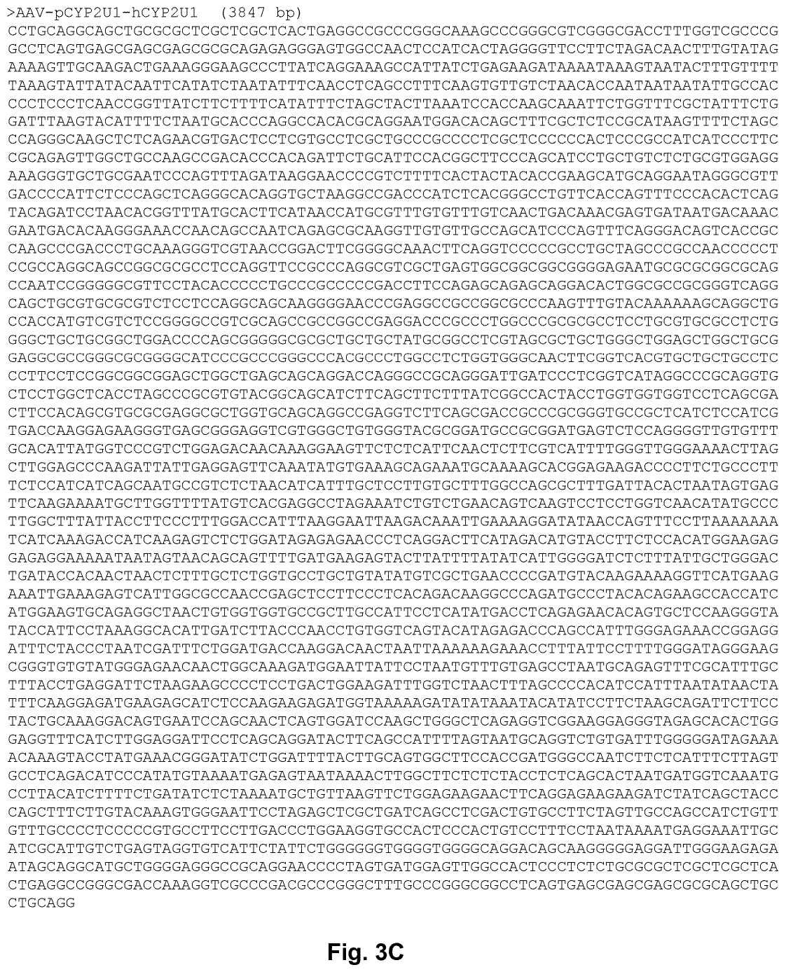

AAV-pEF1a-hCYP2U1. The vector AAV-pEF1a-hCYP2U1 contains the AAV2 inverted terminal repeats joined to an expression cassette in which expression of the cDNA for human CYP2U1 is driven by the promoter of the human EF1a gene to produce a moderate level of gene expression in all cell types. The vector is shown schematically in FIG. 2A and the nucleotide sequence thereof is in FIG. 3A. The genetic elements of this construct are provided in Table 1A. A vector encoding the mouse CYP2U1 homolog was constructed by substituting the mouse cDNA for the human cDNA to produce AAV9-pEF1a-mCYP2U1.

TABLE 1A
Construction of AAV-pEF1a-hCYP2U1

| Nucleotide | Element |
|---|---|
| 1-141 | Left hand ITR of AAV2 (Reverse complement of right ITR) |
| 169-1347 | Human EF1a promoter |
| 1378-3529 | cDNA for human CYP2U1 |
| 1378-3066 | Human CYP2U1 Isoform X1 protein open reading frame |
| 3584-3791 | Bovine growth hormone poly A tail |
| 3799-3939 | Right hand ITR of AAV2 |

AAV-pSYN1-hCYP2U1. The vector AAV-pSYN1-hCYP2U1 contains the AAV2 inverted terminal repeats joined to an expression cassette in which expression of the cDNA for human CYP2U1 is driven by the promoter of the human SYN1 gene to produce gene expression in neurons. The vector is shown schematically in FIG. 2B and the nucleotide sequence thereof is in FIG. 3B. The genetic elements of this construct are provided in Table 1B. A vector encoding the mouse CYP2U1 homolog was constructed by substituting the mouse cDNA for the human cDNA to produce AAV9-pSYN1-mCYP2U1.

TABLE 1B
Construction of AAV-pSYN1-hCYP2U1

| Nucleotide | Element |
|---|---|
| 1-141 | Left hand ITR of AAV2 (Reverse complement of right ITR) |
| 169-637 | Human SYN1 promoter |
| 668-3302 | cDNA for human CYP2U1 |
| 668-2356 | Human CYP2U1 Isoform X1 protein open reading frame |
| 3357-3564 | Bovine growth hormone poly A tail |
| 3572-3712 | Right hand ITR of AAV2 |

AAV-pCYP2U1-hCYP2U1. The vector AAV-pCYP2U1-hCYP2U1 contains the AAV2 inverted terminal repeats joined to an expression cassette of in which expression of the cDNA for human CYP2U1 is driven by its own promoter, the human CYP2U1 promoter, to provide expression in the same cellular pattern as the endogenous CYP2U1 gene. The vector is shown schematically in FIG. 2C and the nucleotide sequence thereof is in FIG. 3C. The genetic elements of this construct are provided in Table 1C. A vector encoding the mouse CYP2U1 homolog was constructed by substituting the mouse cDNA for the human cDNA to produce AAV9-pCYP2U1-mCYP2U1.

TABLE 1C
Construction of AAV-pCYP2U1-hCYP2U1

| Nucleotide | Element |
|---|---|
| 1-141 | Left hand ITR of AAV2 (Reverse complement of right ITR) |
| 169-1255 | Human CYP2U1 promoter |
| 1286-3437 | cDNA for human CYP2U1 |
| 1286-2974 | Human CYP2U1 Isoform X1 protein open reading frame |
| 3492-3699 | Bovine growth hormone poly A tail |
| 3707-3847 | Right hand ITR of AAV2 |

AAV Vector Production. Standard methods for preparing AAV vectors are as described using a three plasmid transfection system in 293T cells (Lukashchuk 2016; Wu 2006; De 2006). These plasmids are the AAV replicon-transgene plasmids encoding the CYP2U1 gene; a first AAV helper plasmid encoding the AAV replication protein (rep) and the serotype-specific AAV capsid protein (cap), in this case the AAV9; and a second AAV helper plasmid encoding the adenovirus E2 and E4 proteins. The adenovirus E1 function is provided by the 293T cells.

Briefly, the three plasmids are transfected into the 293T cells using the PolyFect Transfection Reagent (Qiagen) and cells are grown for 48-72 hours. Cells are harvested and lysed and cellular DNA is removed by DNase1 digest. The lysate is concentrated and loaded onto an iodixanol step gradient. The AAV band is collected and the suspension buffer composition is adjusted to phosphate buffered saline by membrane dialysis. Viral titer is determined by qPCR using primer and probe for inverted terminal repeats (Lukashchuk 2016; De 2006).

### Example 2. Efficacy of AAV SPG56 Vectors in a Mouse Knockout Model

SPG56 knockout mice, namely Cyp2u1^{tm1b(UCOMM)Wtsi} mice (/MGI:1918769; http://www.informatics.jax.org/marker/MGI:1918769), are used to assess the efficacy of CNS delivery and the impact on phenotype with the AAV vectors of Example 1. This strain consists of an insertion of a lacZ expression module after exon 1 and results in a truncation of the mouse CYP2U1 gene. The homozygous insertion resembles a human SPG56 patient with little to no CYP2U1 enzymatic activity. The mouse has been extensively phenotyped by the International Mouse Knockout Consortium (IMPC), (https://www.mousephenotype.org/data/genes/MGI:1918769), revealing partial male embryonic lethality and a defect in grasping responses. Other defects are observed in spontaneous motor activity in open field testing. A thorough examination showed that rotarod, treadmill and gait were unaffected but that there was a significant defect in Y maze test of learning and memory [Pujol 2021]. Also, significant abnormalities of ubiquinone composition in brain were detected as well and morphological deterioration of the retina in aged knockout mice [Pujol 2021].

The mice are administered vector or vehicle by the intracerebroventricular (ICV) route in a single injection at the indicated dosage as described in Table 2 using the cohorts listed in Table 3.

TABLE 2
CYP2U1 Mouse Mutant Efficacy Protocol

| Species/Strain: | CYP2U1^{tm1a(EUCOMM)Wtsi} |
|---|---|
| Breeding | Homozygous male × heterozygous female |
| Administration | Bilateral intra-cerebroventricular on post- |
| | natal day 1. 2 μl per hemisphere of undiluted |
| | vector |
| Vector | Dose: |
| AAV9-pEF1a-mCYP2U1 | 2 × 10¹³ genome copies/ml |
| AAV9-pEF1a-hCYP2U1 | 2 × 10¹³ genome copies/ml |
| AAV9-pSYN1-mCYP2U1 | 2 × 10¹³ genome copies/ml |
| AAV9-pCYP-hCYP2U1 | 2 × 10¹³ genome copies/ml |

TABLE 3
Cohorts for CYP2U1 Mouse Efficacy Study

| Cohort | Genotype | N | Treatment | Vector |
|---|---|---|---|---|
| A | Homozygous female | 10 | None | |
| B | Homozygous female | 10 | Neonatal ICV | AAV9-pEF1a-mCYP2U1 |
| C | Homozygous female | 10 | Neonatal ICV | AAV9-pEF1a-hCYP2U1 |
| D | Homozygous female | 10 | Neonatal ICV | AAV9-pSYN1-mCYP2U1 |
| E | Homozygous female | 10 | Neonatal ICV | AAV9-pCYP-hCYP2U1 |
| F | Wildtype female | 10 | None | |
| G | Homozygous male | 10 | None | |
| H | Homozygous male | 10 | Neonatal ICV | AAV9-pEF1a-mCYP2U1 |
| I | Homozygous male | 10 | Neonatal ICV | AAV9-pEF1a-hCYP2U1 |
| J | Homozygous male | 10 | Neonatal ICV | AAV9-pSYN1-mCYP2U1 |
| K | Homozygous male | 10 | Neonatal ICV | AAV9-pCYP-hCYP2U1 |
| L | Wildtype female | 10 | None | |

For the physical assessments,
whole blood is collected for hematology assessment at 4-5 WOA;
Body weights are measured weekly;
Cage-side observations are recorded daily;
Body composition is determined (UltraFocus DEXA) on days 6, 12 and 18 weeks post AAV injection
Nesting behavior is evaluated over a 24 hour-period at 6 weeks of age (WOA);
Animals are monitored beginning at 7 WOA for five consecutive days using PhenoTyper equipment (Noldus) with video tracking system providing data on general activity over the five-day testing period.

For behavioral assessments, animals are subjected to neuromuscular/behavioral tests at 8 and 16 WOA, including spontaneous activity measured using Open Field, Y-maze spatial recognition, and isometric force measurement.

Upon meeting the end-point criteria or at 18 WOA, animals are humanely euthanized, and necropsy is performed, including gross examination of the following tissues: whole blood, brain, liver, spleen, heart, kidneys, spinal cord, lungs and retina. The collected tissues are processed for H & E staining, histopathology, and assessing CYP2U1 expression levels using RT-PCR. Additionally, hematology and serum chemistry analysis are performed on whole blood and the hippocampus is subjected to lipidomic analysis. The SPG56 knockout mice are known to have higher levels of several quinones than wildtype, including ubiquinol 9, ubiquinone 9, ubiquinol 10 and ubiquinone 10 (Pujol 2021) and can be measured by liquid chromatography and/mass spectrometry (see, e.g., Rousseau 1998).

### Example 3. Mouse Knockout Model Evaluation

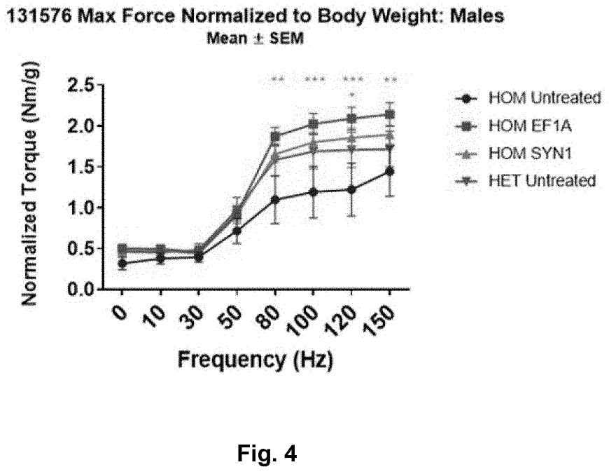

Wildtype mice (HET) and homozygous SPG56 knockout mice (HOM) were evaluated with an in vivo force test to assess muscle force as well as contractile and fatigue characteristics. The muscle force generated by electrical stimulation was higher for a given stimulation frequency in wildtype muscle compared to mutant muscle as seen in FIG. 4 for the torques generated for untreated HET mice (▾) and untreated HOM mice (•), respectively. This result is consistent with the lower limb pathology observed in patients with SPG56 due to CYP2U1 mutations.

Mutant (HOM) and wildtype (HET) mice were also evaluated for a range of serum coenzyme Q forms as shown in Table 4. The differences in serum levels for CoQ8 and CoQ 9 were larger that than previously seen for CoQ10.

Additionally, MM scanning identified an age-dependent accumulation of calcium deposits in the brain, a characteristic found in human patients with several types of hereditary spastic paraplegia.

TABLE 4
Serum Coenzyme Q Isoform Levels

| | CoQ 8 | CoQ 9 | CoQ 10 | CoQ 11 |
|---|---|---|---|---|
| Average ± SD | 0.0096 ± 0.0043 | 0.1326 ± 0.0527 | 0.0440 ± 0.0145 | 0.5323 ± 0.0862 |
| Wildtype (WT) | | | | |
| fmol/mL | | | | |
| Average ± SD | 0.0648 ± 0.0193 | 1.3166 ± 0.4329 | 0.0777 ± 0.0273 | 0.4829 ± 0.0938 |
| Mutant | | | | |
| fmol/mL | | | | |
| Ratio | 6.8 | 9.9 | 1.8 | 0.9 |
| (Mutant/WT) | | | | |
| P value | 0.0002 | 0.0003 | 0.0402 | 0.4116 |

### Example 4. Efficacy of AAV SPG56 Vectors in a Mouse Knockout Model

Neonatal SPG56 knockout mice were injected by intracerebroventricular route with 2×10¹¹ genome copies of the AAV9-pEF1a-mCYP2U1 or the AAV9-pSYN1-mCYP2U1 vectors with expression driven by either a ubiquitous promoter (EF1a) or a neuron-specific promotor (SYN1). For both vectors, gene transfer resulted in relief of the in vivo force defect at 4 and 8 weeks of age in both male and female mice (p<0.05). The AAV vector with the EF1a promoter provided greater benefit than that with the SYN1 promoter (significant at higher stimulation frequency in both male and female mice by post-hoc pairwise test; P<0.01) as depicted in FIG. 4 which shows the torques generated for the homozygous knockout mice treated with vectors where CYP2U1 expression was controlled by the EF1a promoter (▪) or by the SYN1 promoter (▴).

In addition, serum CoQ levels were significantly improved by treatment. At age 14 weeks age, serum CoQ8 levels were 6.8 times higher in homozygous knockout mice relative to wildtype controls and serum CoQ9 levels were 9.9 times higher (P<0.001 both). Neonatal treatment by AAV gene transfer using the ubiquitous EF1a promoter reduced the wildtype/mutant ratio of serum CoQ8 and CoQ9 to 3.4 and 5.0 respectively, towards wildtype levels (P<0.01). However, treatment of homozygous knockouts with AAV driving CYP2U1 expression from the SYN1 promoter did not have a significant impact on the serum CoQ levels compared to untreated controls (P>0.1), consistent with less relief that provided for the in vivo force defect when using the SYN1 promoter.

### REFERENCES

Bibi, F., et al., Rare novel CYP2U1 and ZFYVE26 variants identified in two Pakistani families with spastic paraplegia. J Neurol Sci, 2020. 411: p. 116669.
Citterio, A., et al., Mutations in CYP2U1, DDHD2 and GBA2 genes are rare causes of complicated forms of hereditary spastic paraparesis. J Neurol, 2014. 261(2): p. 373-81.
De, B. P., et al., High levels of persistent expression of alpha1-antitrypsin mediated by the nonhuman primate serotype rh.10 adeno-associated virus despite preexisting immunity to common human adeno-associated viruses. Mol Ther, 2006. 13(1): p. 67-76.
Durand, C. M., et al., CYP2U1 activity is altered by missense mutations in hereditary spastic paraplegia 56. Hum Mutat, 2018. 39(1): p. 140-151.
Egashira, Y., et al., Development of lentiviral vectors for efficient glutamatergic-selective gene expression in cultured hippocampal neurons. Sci Rep, 2018. 8(1): p. 15156.
Fink, J. K., Hereditary spastic paraplegia: clinico-pathologic features and emerging molecular mechanisms. Acta Neuropathol, 2013. 126(3): p. 307-28.
Gadalla, K. K. E., et al., Development of a Novel AAV Gene Therapy Cassette with Improved Safety Features and Efficacy in a Mouse Model of Rett Syndrome. Mol Ther Methods Clin Dev, 2017. 5: p. 180-190.
Gray, S. J., et al., Optimizing promoters for recombinant adeno-associated virus-mediated gene expression in the peripheral and central nervous system using self-complementary vectors. Hum Gene Ther, 2011. 22(9): p. 1143-53.
Guengerich, F. P., M. R. Waterman, and M. Egli, Recent Structural Insights into Cytochrome P450 Function. Trends Pharmacol Sci, 2016. 37(8): p. 625-640.
Kariminejad, A., et al., CYP2U1 mutations in two Iranian patients with activity induced dystonia, motor regression and spastic paraplegia. Eur J Paediatr Neurol, 2016. 20(5): p. 782-7.
Lukashchuk, V., et al., AAV9-mediated central nervous system-targeted gene delivery via cisterna magna route in mice. Mol Ther Methods Clin Dev, 2016. 3: p. 15055.
Masciullo, M., et al., Hereditary spastic paraplegia: Novel mutations and expansion of the phenotype variability in SPG56. Eur J Paediatr Neurol, 2016. 20(3): p. 444-8.
Peviani, M., et al., Lentiviral vectors carrying enhancer elements of Hb9 promoter drive selective transgene expression in mouse spinal cord motor neurons. J Neurosci Methods, 2012. 205(1): p. 139-47.
Pujol, C., et al., Implication of folate deficiency in CYP2U1 loss of function. J Exp Med, 2021. 218(11).
Schüle, R, T. Holland-Letz, S. Klimpe, J. Kassubek, T. Klopstock, V. Mall, S. Otto, B Winner, L Schöls. The Spastic Paraplegia Rating Scale (SPRS): a reliable and valid measure of disease severity. Neurology. 2006 Aug. 8;67(3):430-4.
Sharawat, I. K., P. K. Panda, and L. Dawman, Spastic Paraplegia-56 due to a Novel CYP2U1 Truncating Mutation in an Indian Boy: A New Report and Literature Review. J Pediatr Neurosci, 2021. 16(1): p. 71-74.
Sinnett, S. E., et al., Improved MECP2 Gene Therapy Extends the Survival of MeCP2-Null Mice without Apparent Toxicity after Intracisternal Delivery. Mol Ther Methods Clin Dev, 2017. 5: p. 106-115.
Tesson, C., et al., Alteration of fatty-acid-metabolizing enzymes affects mitochondrial form and function in hereditary spastic paraplegia. Am J Hum Genet, 2012. 91(6): p. 1051-64.
Tornio, A. and J. T. Backman, Cytochrome P450 in Pharmacogenetics: An Update. Adv Pharmacol, 2018. 83: p. 3-32.
Waring, R. H., Cytochrome P450: genotype to phenotype. Xenobiotica, 2020. 50(1): p. 9-18.
Wu, Z., A. Asokan, and R. J. Samulski, Adeno-associated virus serotypes: vector toolkit for human gene therapy. Mol Ther, 2006. 14(3): p. 316-27.
https://www.mousephenotype.org/data/genes/MGI:1918769.

I claim:

1. A nucleic acid encoding an adeno-associated virus (AAV) replicon which comprises in 5′ to 3′ direction (i) a first AAV inverted terminal repeat (ITR), (ii) a promoter operably linked to an CYP2U1 open reading frame, (iii) a polyadenylation (pA) signal operably linked to said open reading frame, and (iv) a second AAV ITR.

2. The nucleic acid of claim 1, wherein the second ITR is the inverse complement of the first ITR.

3. The nucleic acid of claim 1, wherein said promoter is a human or murine EF1a promoter, SYN1 promoter or CYP2U1 promotor.

4. The nucleic acid of claim 1, wherein said CYP2U1 open reading frame encodes a human CYP2U1 protein.

5. The nucleic acid of claim 4, wherein said CYP2U1 open reading frame encodes an CYP2U1 protein having an amino acid sequence of SEQ ID NO: 1.

6. The nucleic acid of claim 1, wherein said CYP2U1 open reading frame encodes a murine CYP2U1 protein.

7. The nucleic acid of claim 1, wherein said pA signal is a human or murine growth hormone pA signal.

8. The nucleic acid of claim 1, wherein said replicon comprises a nucleic acid sequence of any one of those in SEQ ID NOS: 2-4.

9. The nucleic acid of claim 1, wherein said replicon is in a plasmid.

10. The nucleic acid of claim 1, wherein said ITRs are from AAV serotype 2 or a neurotropic AAV serotype.

11. A recombinant AAV (rAAV) comprising AAV capsid proteins or AAV pseudocapsid proteins and the replicon of claim 1 packaged therein.

12. The rAAV of claim 11, wherein said capsids are from AAV serotype 9 or a neurotropic AAV serotype.

13. A pharmaceutical composition comprising an rAAV of claim 11 and a pharmaceutically acceptable carrier.

14. A method for treating or ameliorating one or more symptoms of spastic paraplegia which comprises administering the composition of claim 13 to a subject in an amount and for a time sufficient to treat or ameliorate the one or more symptoms of spastic paraplegia in said subject.

15. The method of claim 14, wherein said subject is a human.

16. The method of claim 15, wherein said pharmaceutical composition comprises an rAAV wherein the replicon packaged therein comprises a human EF1a promoter operably linked to a human CYP2U1 open reading frame and said capsids are from AAV serotype 9 or a neurotropic AAV serotype.

17. The method of claim 14, wherein said composition is administered into cerebrospinal fluid through lumbar puncture, or via intra cisterna magna, intracerebroventricular or intravenous routes.

18. The method of claim 14, wherein spastic paraplegia is spastic paraplegia 56 (SPG56).

19. A method of gene transfer for treating spastic paraplegia or for ameliorating one or more symptoms of spastic paraplegia which comprises administering an rAAV of claim 11 to a mammal in an amount and for a time sufficient to treat said spastic paraplegia or to ameliorate at least one or more symptoms of spastic paraplegia in said mammal.

20. The method of claim 19, wherein spastic paraplegia is spastic paraplegia 56 (SPG56).

21. A method of determining efficacy of a therapeutic agent in treating a spastic paraplegia or a disease associated with a cytochrome P450 defect which comprises
(a) administering a candidate therapeutic agent to a subject;
(b) monitoring a level of one or more Coenzyme Q isoforms in a bodily fluid of said subject relative to a level in said subject prior to administering said agent,
wherein a decrease in the level of one or more Coenzyme Q isoforms relative to baseline indicates said agent is effective for treating said spastic paraplegia or said disease.

22. The method of claim 21, wherein said one or more Coenzyme Q isoform is Coenzyme Q8, Coenzyme Q9 or both.

23. The method of claim 21, wherein said therapeutic agent is an AAV gene therapy vector and delivers a good copy of a gene associated with said spastic paraplegia.

24. A method for treating or ameliorating spastic paraplegia or a disease associated with cytochrome P450 defects which comprises:
(a) measuring a baseline level of one or more Coenzyme Q isoforms in a bodily fluid of a subject having a spastic paraplegia or a disease associated with a cytochrome P450 defect;
(b) administering a therapeutic agent to said subject;
(c) measuring a post-administration level of one or more Coenzyme Q isoforms in a bodily fluid of said subject, wherein a decrease in one or more Coenzyme Q isoforms relative to baseline indicates said spastic paraplegia or said disease associated with a cytochrome P450 defect is responding to said therapeutic agent.

25. The method of claim 24, wherein said spastic paraplegia is a hereditary spastic paraplegia or sporadic spastic paraplegia.

26. The method of claim 25, wherein said spastic paraplegia is selected from the group consisting of SPG3A, SPG4, SPG5A, SPG7, SPG10, SPG11, SPG13, SPG19, SPG26, SPG28, SPG31, SPG35, SPG39, SPG42, SPG46, SPG56, SPG66, SPG67, SPG81, SPG, 81, SPG82, SPG84 and SPG86.

27. The method of claim 24, wherein said spastic paraplegia is SPG56.

28. The method of claim 24, wherein said a cytochrome P450 is CYP2U1.

29. The method of claim 24, wherein said one or more Coenzyme Q isoform is Coenzyme Q8, Coenzyme Q9 or both.

30. The method of claim 24, wherein said bodily fluid is peripheral blood, serum, plasma, ascites, urine, saliva or cerebrospinal fluid (CSF).
